(19) United States
(12) Patent Application Publication
(10) Pub. No.: US 2010/0054491 A1
(43) Pub. Date: Mar. 4, 2010

(54) NOISE-CANCELING HEADSET FOR A CHILD

(76) Inventor: Alexandria R. Griffin, Waxahachie, TX (US)

Correspondence Address: Michael L. Diaz;Diaz and Associates, Suite 200, 555 Republic Drive, Plano, TX, 75074, US

(21) Appl. No.: 12/198,745

(22) Filed: Aug. 26, 2008

(51) Int. Cl. A61F 11/06

(52) U.S. Cl. 381/71.6; 381/374

(57) ABSTRACT

A headset for use by a child, such as an infant or small child. The headset includes a front headband sized and shaped to fit over a front portion of the head of the child and a rear headband sized and shaped to fit over a rear portion of the child's head and coupled to the front headband. The headset also includes a first ear cover attached to one side of the front headband and a second ear cover attached to an opposite second side of the front headband. The ear covers each include a noise-canceling earpiece. The headset is positioned over the head of the child and the ear covers are position over the ears of the child to provide noise cancelation to the child.

### BACKGROUND OF THE INVENTION

1. Field of the Invention

This invention relates to audio equipment. Specifically, and not by way of limitation, the present invention relates to a noise-canceling headset for children.

2. Description of the Related Art

Many infants and children are subjected to high levels of noise which is detrimental to their hearing. In particular, infants travel on aircraft (both commercial and general aviation), where the noise level is oftentimes at a level which is unsafe for the infant. However, ordinary means of reducing the noise level is not satisfactory for infants or small children. Adults often use earplugs to reduce the noise level. These earplugs must be inserted into the ear canal to be effective. However, these earplugs are uncomfortable for infants to wear and provide a problem to the parents in keeping the earplugs in the ears. In addition, it has been recommended by physicians not to insert devices into the ear canals of young children. Adults also use audio headsets or noise-canceling heads to remove ambient noise or play music over the ambient noise. However, the headsets require also must be inserted into the ear. Alternately, large bulky headphones are used by adults to cancel the noise. These headphones are unusable by infants because they would not remain on the infant's head.

It would be advantageous to have a noise-canceling headset which is comfortable for wear by a child and provide a noise-canceling feature to the child's ears. It is an object of the present invention to provide such an apparatus.

### SUMMARY OF THE INVENTION

In one aspect, the present invention is directed to a headset for use by a child, such as an infant. The headset includes a front headband sized and shaped to fit over a front portion of the head of a child and a rear headband sized and shaped to fit over a rear portion of the child's head and coupled to the front headband. The headset also includes a first ear cover attached to one side of the front headband and a second ear cover attached to an opposite second side of the front headband. The ear covers each include a noise-canceling earpiece. The headset is positioned over the head of the child and the ear covers are position over the ears of the child to provide noise cancelation to the child. The headbands may include adjustment straps for adjusting the length of the headbands.

In one embodiment, the noise-canceling earpieces each include cushioned perimeters to raise the earpieces away from the child's ears.

In another embodiment, the noise-canceling earpieces are removable from the ear covers and include a headband slot for retaining a conventional headband.

In another embodiment, the noise-canceling earpieces are removable from the ear covers and include an interphone jack for coupling to an audio system.

In another aspect, the present invention is directed to a headset retention apparatus for use by a child. The headset retention apparatus includes a front headband sized and shaped to fit over a front portion of a head of a child, a rear headband sized and shaped to fit over a rear portion of the head of the child and coupled to the front headband. In addition, the headset retention apparatus includes a first ear cover attached to one side of the front headband and a second ear cover attached to an opposite second side of the front headband. The ear covers are used for retaining noise-canceling earpieces.

### BRIEF DESCRIPTION OF THE DRAWINGS

FIG. 1 is a side view of a noise-canceling headset worn by a child in the preferred embodiment of the present invention;

FIG. 2 is a front view of the headset of FIG. 1;

FIG. 3 is a side view of the ear cover of FIG. 1;

FIG. 4 is a side inner view of the earpiece in the preferred embodiment of the present invention;

FIG. 5 is a outer side view of the earpiece in an alternate embodiment of the present invention; and

FIG. 6 is a side view of the earpiece of FIG. 5 for use by an older child.

### DESCRIPTION OF THE INVENTION

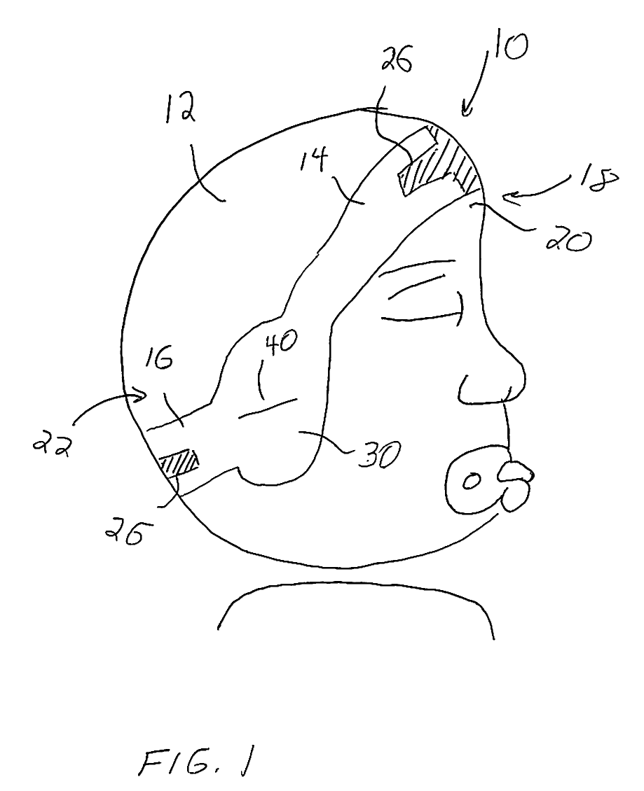

The present invention relates to a noise canceling headset for use by children, such as infants and small children. FIG. 1 is a side view of a noise-canceling headset 10 worn by an infant 12 in the preferred embodiment of the present invention. The headset includes a front headband 14 and a rear headband 16. The front headband preferably is sized and shaped to arch in front of an upper portion 18 of the forehead 20 of the infant. The rear headband 16 is sized and shaped to fit about a rear portion 22 of the infant above the neck. The front and rear headbands are preferably adjustable by straps 26. An adjustable strap may be used, such as hook and pile material, Velcro-less material, etc. In one embodiment, the inner surface of the front headband 14 and the rear headband 16 may include silicon strips to prevent slippage of the headbands on the infant's head. The headbands are preferably constructed of a breathable soft, washable fabric. However, any flexible material may be used and still remain in the scope of the present invention. It should be understood that the present invention may be utilized by any child (i.e., from birth to the onset of adulthood). However, as depicted in FIG. 1, the headset is particularly useful for an infant or small child. As defined for the present invention, a child is any person ranging in age from birth to the onset of adulthood.

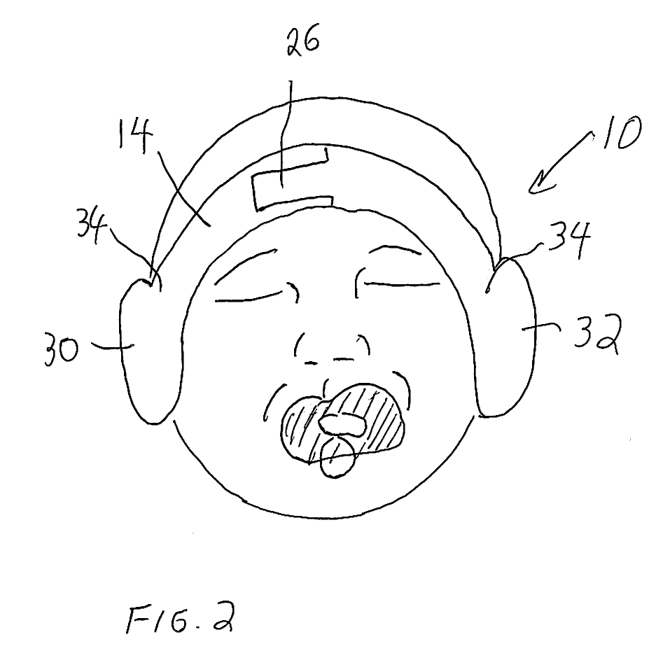

FIG. 2 is a front view of the headset 10 of FIG. 1. As depicted in FIG. 2, the headset includes two ear covers 30 and 32 attached on each end 34 of the front headband 14. In addition, the ear covers are attached to the rear headband 16. The ear covers are sized, shaped and positioned to comfortable fit over the infant's ears.

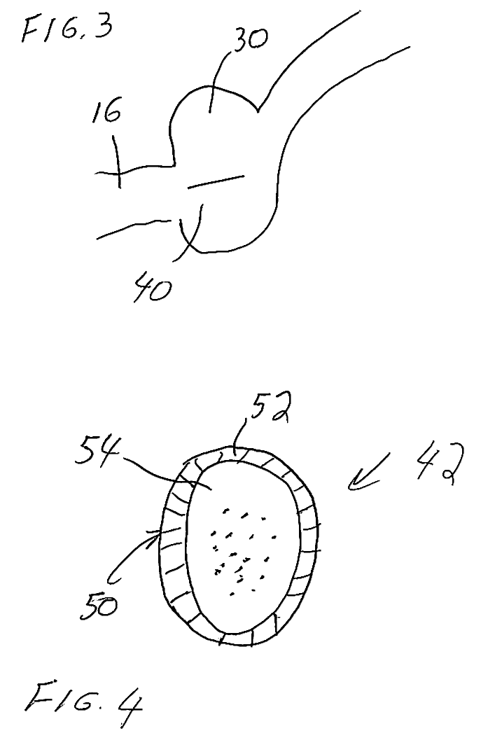

FIG. 3 is a side view of the ear cover 30 of FIG. 1. In the preferred embodiment of the present invention, the ear cover includes a closeable opening 40, such as a slit, for insertion of a noise-canceling earpiece 42 (see FIG. 4). The noise-canceling earpiece 42 is preferably a wireless earphone providing a noise-canceling feature to the user. The use of noise-canceling wireless earpieces is well known in the art. However, in the present invention, a conventional noise-canceling earpiece is configured for use by an infant. The earpiece may be inserted within the ear cover. The ear cover preferably includes a pouch for holding the noise-canceling earpiece in place. Alternately, the earpiece may be integrated directly into the ear cover without the use of a pouch. A wireless noise-canceling earpiece is preferred to prevent the infant from pulling on any attached cords. In addition, in the preferred embodiment of the present invention, the earpiece includes a rechargeable battery. The present invention utilizes two earpieces 42, one for each ear cover, thereby covering both ears of the infant.

FIG. 4 is a side inner view of the earpiece 42 in the preferred embodiment of the present invention. The earpiece is preferably small and light having a substantially oval shape. Preferably, on an outer perimeter 50 of the earpiece is a thin cushion 52. This thin cushion lifts the earpiece off the ear to prevent pressure and discomfort to the infant. The earpiece 42 also includes an interior portion 54. At a junction where the earpiece sits, a thin bendable semi-rigid material may be used to provide a sturdy support for the earpiece as well as provide an adjustment to the ear cover to accommodate placement of the earpiece 42 over the infant's ear. The thin bendable semi-rigid material may be integrated into the headband and project towards the earpiece in order to mold the earpiece to the ear for a firmer fit.

The use of two headbands allow the comfortable positioning and retention of the ear covers on the infant's head. By positioning the front headband above the forehead and the rear headband on a rear portion of the infant's neck, the ear covers stay in place over the infant's ears despite any movement by the infant. In addition, the ear covers provide comfortable placement of the noise-canceling earpieces 42 over the infant's ear, enabling use of the noise-canceling feature without inserting the earpieces in the infant's ear canals. The headbands are both adjustable, which enable the headset 10 to be used children of various ages. Thus, the headbands may be loosened to accommodate a larger head, thereby being useable by the infant as the infants grows bigger. In addition, the earpieces 42 may be removed from the ear covers and the headset may be washed. In an alternate embodiment of the present invention, the noise-canceling earpieces may be receivers for receiving broadcasts from the aircraft's radio or music from a music playing device.

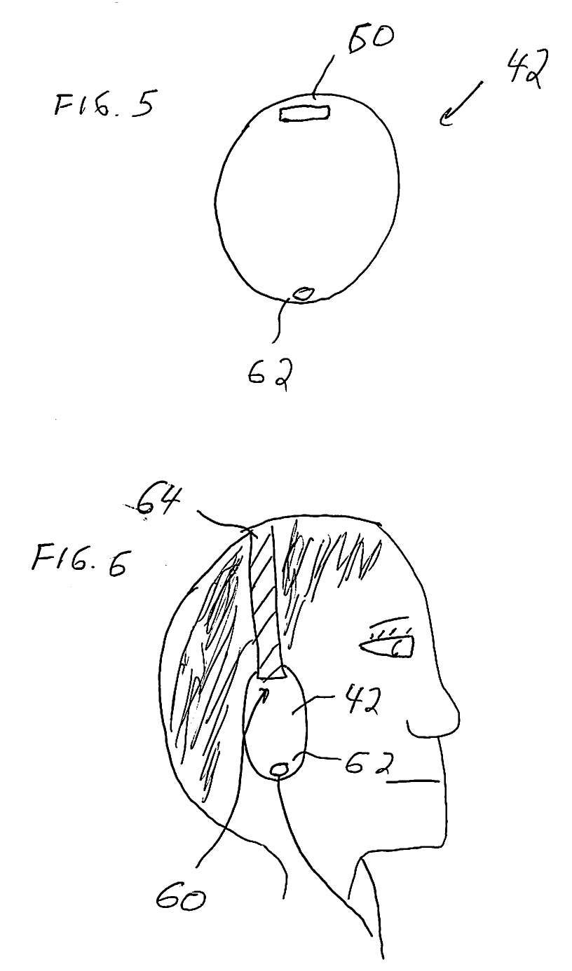

FIG. 5 is an outer side view of the earpiece 42 in an alternate embodiment of the present invention. The earpiece may include a headband slot 60 and/or an interphone jack 62. The earpiece having the noise-canceling feature (not shown) may be utilized by an older child for which the front and rear headbands 14 and 16 are not needed. In such a situation, the child may utilize a conventional headband 64. The headband 64 is a conventional headband having a semi-rigid material spanning over the child's head from one ear to the other ear. The earpieces 42 are removed from the ear covers and attached to the headband 64. The ear covers may then be connected to an aircraft interphone system or other audio device via a wire or wires 66.

FIG. 6 is a side view of the earpiece 42 of FIG. 5 for use by an older child. The earpiece includes the headband slot 60 and/or an interphone opening 62. The headband slot 60 allows the headband 64 to be positioned in the ear cover. The interphone opening 62 allows the wire 66 to be inserted into the earpiece. The headband may be connected to the earpiece by a slot retaining the headband. The wire 66 may include a plug for inserting into a jack 62 of the earpiece.

The present invention, when utilized by an older child, as depicted in FIGS. 5 and 6, enable the child to connect to the aircraft's intercom system and listen to the radio. Alternately, the earpieces may be utilized to connect to any other wireless or wired audio devices. Thus, the present invention may be utilized by a child of a wide range of years, from an infant to an older child or teenager.

The present invention provides a comfortable device enabling an infant to utilize an aviation noise-canceling device which reduces ambient noise in an aircraft. The present invention is preferably wireless to prevent the infant from pulling any wires. However, in alternate devices, the present invention may be connected via a cable to an audio device or noise cancelation system. In addition, the present invention provides an adjustment mechanism (i.e., dual headbands), which enables the headset to be comfortably held on the head of the infant. The headbands are adjustable to fit heads of various sizes. In addition, the present invention is modifiable to be used by older children. Thus, the present invention is quite versatile providing a headset for a child, ranging from birth to the onset of adulthood. Additionally, the present invention may be used in any noisy environment and is not limited to use on an aircraft.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

Thus, the present invention has been described herein with reference to a particular embodiment for a particular application. Those having ordinary skill in the art and access to the present teachings will recognize additional modifications, applications and embodiments within the scope thereof.

It is therefore intended by the appended claims to cover any and all such applications, modifications and embodiments within the scope of the present invention.

What is claimed is:

1. A headset for use by a child, the headset comprising:
a front headband sized and shaped to fit over a front portion of a head of the child;
a rear headband sized and shaped to fit over a rear portion of the head of the child and coupled to the front headband; and
a first ear cover attached to one side of the front headband and a second ear cover attached to an opposite second side of the front headband;
the first ear cover includes a first noise-canceling earpiece and the second ear cover includes a second noise-canceling earpiece;
whereby the headset is positioned over the head of the child and the ear covers are position over ears of the child to provide noise cancelation to the child.

2. The headset according to claim 1 wherein the front headband includes an adjustment strap for adjusting a length of the front headband.

3. The headset according to claim 1 wherein the rear headband includes an adjustment strap for adjusting a length of the front headband.

4. The headset according to claim 1 wherein:
the first ear cover includes a first pouch for holding the first noise-canceling earpiece; and
the second ear cover includes a second pouch for holding the second noise-canceling earpiece.

5. The headset according to claim 4 wherein:
the first pouch includes a first closeable opening for inserting the first earpiece into the first pouch; and
the second pouch includes a second closeable opening for inserting the second earpiece into the second pouch.

6. The headset according to claim 1 wherein the headset is constructed of a washable fabric.

7. The headset according to claim 1 wherein:
the first noise-canceling earpiece includes a first cushion perimeter; and
the second noise-canceling earpiece includes a second cushion perimeter.

8. The headset according to claim 1 wherein the first and second noise-canceling earpieces are wireless and rechargeable.

9. The headset according to claim 1 wherein:
the first and second noise-canceling earpieces are removable from the first and second ear covers;
the first and second noise canceling earpieces each include a headband slot for retaining a conventional headband.

10. The headset according to claim 1 wherein:
the first and second noise-canceling earpieces are removable from the first and second ear covers;
the first and second noise canceling earpieces each include an interphone jack adapted for coupling to an aviation intercom system.

11. The headset according to claim 1 wherein:
the first and second noise-canceling earpieces are removable from the first and second ear covers;
the first and second noise canceling earpieces each include an interphone jack adapted for coupling to an audio system.

12. A headset retention apparatus for use by a child, the headset retention apparatus comprising:
a front headband sized and shaped to fit over a front portion of a head of the child;
a rear headband sized and shaped to fit over a rear portion of the head of the child and coupled to the front headband; and
a first ear cover attached to one side of the front headband and a second ear cover attached to an opposite second side of the front headband;
the first and second ear covers include means for retaining noise-canceling earpieces;
whereby the headset retention apparatus is positioned over the head of the child and the ear covers are position over ears of the child to provide noise cancelation to the child when earpieces are positioned in the ear covers.

13. The headset retention apparatus according to claim 12 wherein the front headband includes an adjustment strap for adjusting a length of the front headband.

14. The headset retention apparatus according to claim 12 wherein the rear headband includes an adjustment strap for adjusting a length of the front headband.

15. The headset retention apparatus according to claim 12 wherein:
the first ear cover includes a first pouch for holding a noise-canceling earpiece; and
the second ear cover includes a second pouch for holding a noise-canceling earpiece.

16. The headset retention apparatus according to claim 15 wherein:
the first pouch includes a first closeable opening for inserting an earpiece into the first pouch; and
the second pouch includes a second closeable opening for inserting an earpiece into the second pouch.

17. The headset retention apparatus according to claim 12 wherein the headset retention apparatus is constructed of a washable fabric.
